(19) United States
(12) Patent Application Publication
(10) Pub. No.: US 2024/0366122 A1
(43) Pub. Date: Nov. 7, 2024

(54) LC-CIRCUIT BASED ELECTRONICS FOR DETECTION OF MULTIPLE BIOMARKERS IN BODILY FLUIDS

(71) Applicant: OHIO STATE INNOVATION FOUNDATION, Columbus, OH (US)

(72) Inventors: Jinghua Li, Columbus, OH (US); Tzu-Li Liu, Columbus, OH (US)

(21) Appl. No.: 18/285,513

(22) PCT Filed: Apr. 6, 2022
(86) PCT No.: PCT/US2022/023668

§ 371 Date: Oct. 4, 2023

Related U.S. Application Data
(60) Provisional application No. 63/171,694, filed on Apr. 7, 2021.

(51) Int. Cl. A61B 5/145; A61B 5/00; A61B 5/1486

(52) U.S. Cl. CPC A61B 5/14546; A61B 5/1451; A61B 5/14517; A61B 5/1486; A61B 5/6802; A61B 5/6847; A61B 2560/0204; A61B 2560/0223; A61B 2560/0443; A61B 2560/0468; A61B 2562/0215; A61B 2562/227

(57) ABSTRACT

Wireless, lightweight, and multifunctional chemical sensors and methods for detection of biomarkers in bodily fluids are described. Systems and methods are directed to an LC (inductor-capacitor) resonance circuit configured to detect multiple biomarkers in bodily fluids of a subject through frequency modulation. The system may be comprised within a wearable device or within a medical implant, depending on the implementation. The body fluid may comprise sweat, cerebrospinal fluid, blood, saliva, tears, mucus, gastric acid, and/or urine, for example. The biomarkers may comprise Na+, K+, Ca2+, H+, Cl—, glucose, urea, lactate, glutamate, serotonin, cortisol, dopamine, cytokines, and/or epinephrine, for example.

### CROSS-REFERENCE TO RELATED APPLICATIONS

This application claims the benefit of U.S. provisional patent application No. 63/171,694, filed on Apr. 7, 2021, and entitled “LC-CIRCUIT BASED ELECTRONICS FOR DETECTION OF MULTIPLE BIOMARKERS IN BODILY FLUIDS,” the disclosure of which is expressly incorporated herein by reference in its entirety.

### BACKGROUND

Tracking concentration of biomarkers in biofluids can provide crucial information about health status. However, the complexity and non-ideal form factors of conventional digital wireless schemes impose challenges in realizing bio-integrated, lightweight, and miniaturized sensors.

More particularly, the levels of multiple biochemical components such as electrolytes, metabolites, neurotransmitters, and hormones in bodily fluids, can serve as important biomarkers for the monitoring of physiological or psychological status. Rapid and accurate detection of these biomarkers in bodily fluids (e.g., sweat, cerebrospinal fluid, etc.) using wireless electronics represents an important approach in personalized, precision medicine. However, the accurate quantification of these biomarkers with high sensitivity and selectivity remains challenging. Additionally, conventional digital wireless schemes compatible with electrochemical sensing technologies require the use of bulky, integrated electronic modules and batteries, which impose limitations for miniaturizing the sensing device and tracking subjects' health status without interfering with their normal daily activities.

Moreover, the grand societal challenges regarding the ever-increasing demand for and cost of health care have motivated continued research efforts to develop personalized, precise, and preventive medicine. To this end, bio-integrated sensing technologies play significant roles in monitoring patients' vital signs and detecting important biological anomalies. While commercial wearable and implantable sensors have evolved in recent years in monitoring various biophysical signals, the sensitive, selective, and wireless detection of chemical biomarkers in bodily fluids is worth further study to obtain insight into physiological and pathological pathways at molecular levels. Hundreds of biomarkers exist in bodily fluids which could provide abundant information for biomedical research and clinical practices. For example, sweat is a highly attractive candidate due to the easy accessibility and the rich biochemical information it carries. The level of multiple electrolytes and metabolites can serve as biomarkers for the monitoring of physical and emotional health status. Similarly, the accurate detection of neurotransmitters and hormones is one of the most promising techniques in understanding brain functions. Consequently, developing wireless and bio-integrated sensing technologies could potentially shift the paradigm of healthcare from a slow, centralized, and generic mode to a rapid, low-cost, and personalized one.

It is with respect to these and other considerations that the various aspects and embodiments of the present disclosure are presented.

### SUMMARY

Wireless, lightweight, and multifunctional chemical sensors and methods for detection of biomarkers in bodily fluids are described. Systems and methods are directed to an LC (inductor-capacitor) resonance circuit configured to detect multiple biomarkers in bodily fluids of a subject through frequency modulation. The system may be comprised within a wearable device or within a medical implant, depending on the implementation. The body fluid may comprise sweat, cerebrospinal fluid, blood, saliva, tears, mucus, gastric acid, and/or urine, for example. The biomarkers may comprise Na⁺, K⁺, Ca²⁺, H⁺, Cl⁻, glucose, urea, lactate, glutamate, serotonin, cortisol, dopamine, cytokines, and/or epinephrine, for example.

In an implementation, a system comprises: a sensing interface comprising an inductor-capacitor (LC) circuit configured to detect multiple biomarkers in bodily fluids of a subject; a coupling unit; and a stretchable wire connecting the sensing interface and the coupling unit.

In an implementation, a system comprises: a receiver and a transducer configured to detect multiple biomarkers simultaneously in body fluid of a subject and monitor a health status of the subject without a battery and configured to transmit data wirelessly.

In an implementation, a method comprises: determining an electric potential change in a DC part of a LC resonance chemical sensor; determining a capacitance modulation in an AC part; and determining a resonance frequency shift in the AC part.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is a schematic illustration of a stretchable, battery-free system for use in a sensor system in detecting various biomarkers (e.g., ion, neurotransmitters, hormones, metabolites, growth factors, etc.) in bodily biofluids;

FIG. 2 is a schematic illustration of the relative position of the sensing platform and the readout coil;

FIG. 3 shows an equivalent circuit and flowchart for the signal conversion and transmission process of the sensor system;

FIG. 4 illustrates a diagram of an implementation of a method for detection of biomarkers in bodily fluids;

FIG. 5 illustrates a diagram of an implementation of a system for detection of biomarkers in bodily fluids;

FIG. 6 is a schematic illustration of the DC part of ion-selective membrane-functionalized sensors, and the interaction between ion-selective membranes and ions at the solution-sensor interface;

FIG. 7 shows a schematic illustration of the interaction between serotonin and an anti-serotonin aptamer-functionalized Au surface;

FIG. 8 shows a schematic illustration of the biofuel cell-inspired biochemical interface for glucose sensing;

FIG. 9 is a photograph of a multiplexed sensor system based on the LC circuit and sensing performance in mixed ion solutions;

FIG. 10 illustrates a diagram of an implementation of a clasp of a “smart necklace” in an open state that may be worn to monitor a subject's biomarkers in bodily fluids;

FIG. 11 illustrates a diagram of an implementation of a clasp of a “smart necklace” in a closed state that may be worn to monitor a subject's biomarkers in bodily fluids;

FIG. 12 illustrates a diagram of an implementation of a wearable device (e.g., a pendant of a “smart necklace”) that may be worn by a subject to monitor the subject's biomarkers in bodily fluids;

FIG. 13 illustrates a diagram of an implementation of a wearable device (e.g., a pendant) that may be worn by a subject to monitor the subject's biomarkers in bodily fluids;

FIGS. 14A, 14B, 14C, and 14D illustrate diagrams of an implementation of a “smart necklace”;

FIG. 15 illustrate a diagram of an implementation of an implantable sensor that may be used to monitor a subject's biomarkers;

FIG. 16A shows an exploded view of an as-prepared probe;

FIGS. 16B and 16C show conceptual illustrations of a potential application where the coupling unit is embedded in the skull to provide stable signal transmission while the sensing interface extends to the area with cerebrospinal fluid (CSF) or brain tissues of interest; and

FIG. 17 shows an exemplary computing environment in which example embodiments and aspects may be implemented.

### DETAILED DESCRIPTION

This description provides examples not intended to limit the scope of the appended claims. The figures generally indicate the features of the examples, where it is understood and appreciated that like reference numerals are used to refer to like elements. Reference in the specification to “one embodiment” or “an embodiment” or “an example embodiment” means that a particular feature, structure, or characteristic described is included in at least one embodiment described herein and does not imply that the feature, structure, or characteristic is present in all embodiments described herein.

In recent years, studies on developing novel biochemical sensors and/or integrated systems with supporting wireless modules have received considerable attention and are progressing rapidly. The success and breakthroughs of pioneering works in this field have set the stage for next-generation healthcare. Nevertheless, the following issues warrant further investigation. Most currently available digital wireless schemes compatible with chemical sensing technologies require the use of integrated electronic components (e.g., operational amplifier, microcontrollers, etc.) for signal generation/transmission and batteries/energy harvesting systems for power supply, with conventional rigid printed circuit boards serving as mounting sites. The non-ideal form factors, including the size, weight, and rigidity of the subsystems may constrain natural motions. The high level of complexity of the circuit layouts also imposes practical challenges for seamless integration with skin/other bio-tissues and precise interpretation of experimental results. In this context, passive resonance circuits are of great interest due to the simple, lightweight, battery-free, and miniaturizable structure suitable for wireless wearable/implantable applications. In these systems, target signals modulate a responsive element (i.e., inductance, capacitance, or resistance) in the circuit, which then results in a change in the characteristics around the resonance peak (i.e., amplitude and/or frequency). Consequently, it is highly desirable to develop sensing systems and methods using resonance circuitry for detection of various biomarkers. In addition to the wireless sensing capability, desired key performance metrics may include sensitivity, selectivity, and multifunctionality to support concurrent recording of multiple biochemical signals with a minimal level of crosstalk for establishing biometric signature profiles associated with health status. In the context of wearable/implantable applications, how to minimize motion/strain/environment-induced changes in the electrical performance of the circuit warrants further attention. Overall, addressing these issues uses joint efforts in the proper design of both the biochemical interfaces and the electronic transducers, as well as the coupling strategy between them.

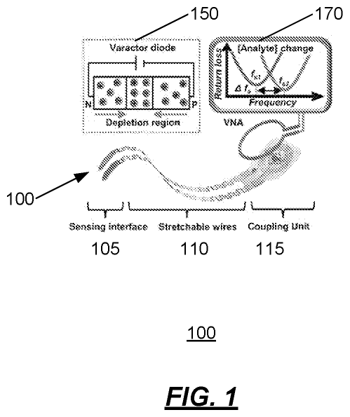

FIG. 1 is a schematic illustration of a stretchable, battery-free system 100 for use in a sensor system in detecting various biomarkers (e.g., ion, neurotransmitters, hormones, metabolites, growth factors, etc.) in bodily biofluids (e.g., CSF, tears, saliva, ISF, urine, etc.). The system 100 comprises a stretchable biochemical sensing interface 105 (direct current (DC) part) to be in contact with target biofluids, and coupling unit 115 (e.g., a loop antenna (alternating current (AC) part)) for wireless signal transmission. Stretchable wires 110 connect the sensing interface 105 to the coupling unit. The sensing interface 105 may comprise a varactor diode 150 and the coupling unit 115 may be used to output data to determine a biomarker change 170.

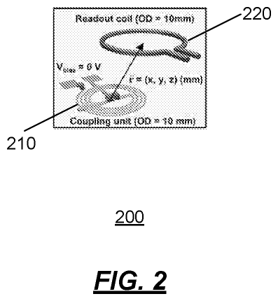

FIG. 2 is a schematic illustration 200 of the relative position of the sensing platform 210 and the readout coil 220.

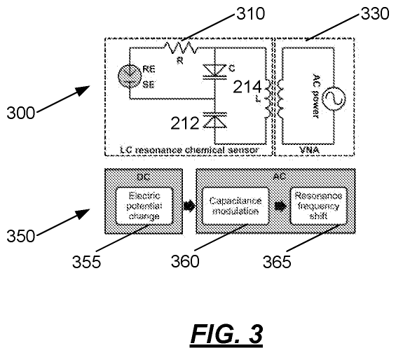

FIG. 3 shows an equivalent circuit 300 and flowchart 350 for the signal conversion and transmission process of the sensor system 100. A sensor 310, such as a LC resonance chemical sensor is a DC part and is connected to a vector network analyzer (VNA) 330 which is an AC component, using stretchable wires. Stretchable wires connecting the DC and AC parts (the sensor 310 and the VNA 330, respectively) can effectively distribute mechanical strains and protect functional components from deformations. An electric potential change 355 in the DC part results in capacitance modulation 360 and resonance frequency shift 365 in the AC part.

More particularly, the working principle of tuning circuits in RF electronics inspires the design based on the resonance between an inductor 314 and a pair of varactor diodes 312: the interaction between analytes and the corresponding bio-recognition elements creates a potential difference across the two electrodes in the DC part that scales with the concentration of target biochemical species. The potential serves as the reverse bias for the two series varactor diodes 312. The varactor diodes 312 then convert the change in potential into an alternation in the capacitance of the diodes by modifying the thickness of the depletion region in the p-n junctions. The head-to-head (or back-to-back) varactor configuration eliminates the DC path through the inductor 314 and prevents AC signals from modulating the DC voltage by cancelling out RF voltage induced capacitance variation. When needed, one varactor can also be replaced with a capacitor. The modulation in capacitance induced by the bias voltage can be characterized by measuring fₛ of the resonance circuit according to equation (1):

$${f}_{s} = \frac{1}{2 π \sqrt{LC}} \qquad ( 1 )$$

where L and C are the inductance and capacitance of the resonance circuit, respectively. Coupling the inductor 314 to an alternating magnetic field through a readout coil enables the recording of the input return loss (S₁₁) using the VNA 330. Characterizing the signal reflectance magnitude (S₁₁ in dB) yields a dip around fₛ, and fitting the curve estimates the value of fₛ.

An implementation may use commercial silicon hyper-abrupt junction varactor diodes (Skyworks SMV-1249, 1.0×0.6×0.46 mm³) due to their high voltage sensitivity (i.e., variation of C with reverse bias). Applying a reverse bias to the diodes allows for the evaluation of the electrical performance of the LC circuit in transducing DC signals.

Simulation results were obtained using the loop antenna FIG. 1 with a DC reverse bias ranging from 200 to 400 mV (step: 50 mV). The LC circuit has a fₛ of ˜110 MHz and a Q factor of ˜15. With an increasing reverse bias, the width of the depletion layer increases. That leads to a decrease in the capacitance in the LC circuit which then results in an increasing fₛ according to equation (1). The calibration functionfs (V) is near-linear when the reverse bias is small and in the range of physiologically meaningful signals caused by bio-recognition events (typically <200 mV). The system can clearly distinguish a difference in the input voltage of as low as 1 mV, indicative of the capability of the system in detecting minute changes in biochemical signals. Although some implementations use hyper-abrupt junctions to achieve a high voltage sensitivity, other types of pn and Schottky junction diodes can also work for this circuit model based on bias voltage induced capacitance change and frequency modulation of the resonance circuit.

FIG. 4 illustrates a diagram of an implementation of a method 400 for detection of biomarkers in bodily fluids. The method 400 may be implemented using a sensor 410, a coupling unit 420, and a computer 440. The computer (computing device) 440 may be implemented using a variety of computing devices such as desktop computers, laptop computers, tablets, etc. Other types of computing devices may be supported. A suitable computing device is illustrated in FIG. 17 as the computing device 1700.

At 412, a potentiometric sensor of the sensor 410 senses one or more biomarkers. At 414, a potential difference between SE and RE is determined by the sensor 410.

At 422, a capacitance change is determined by the coupling unit 420. At 424, a resonant frequency (RF) shift is determined by the coupling unit 420.

At 430, the RF shift is provided (e.g., via a network or wireless communication 425, for example) to the computer 440 for calibrated concentration determination.

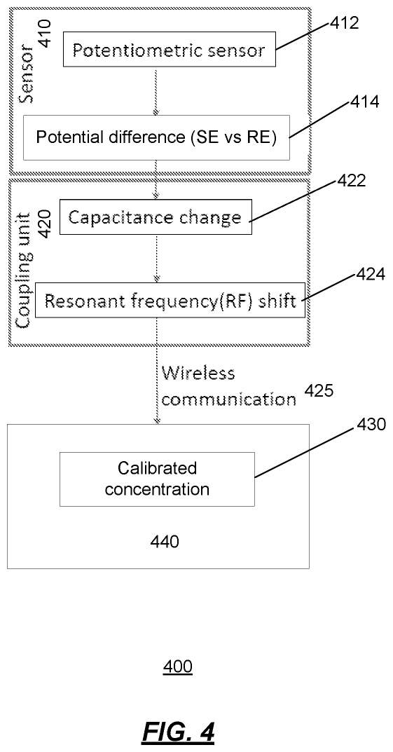

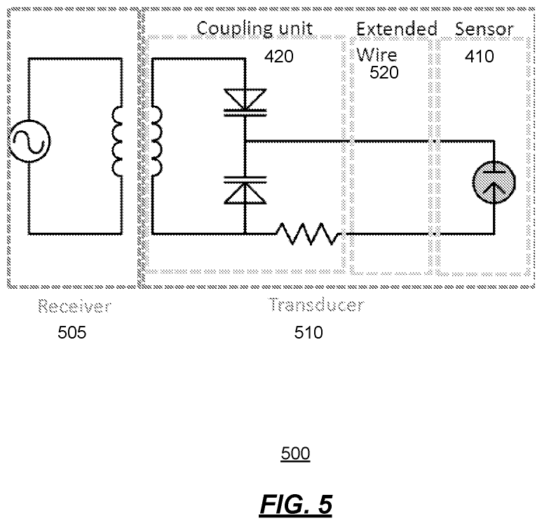

FIG. 5 illustrates a diagram of an implementation of a system 500 for detection of biomarkers in bodily fluids. The system 500 may be used to perform at least some of the operations described herein. The system 500 comprises a receiver 505 and a transducer 510. The transducer 610 comprises the sensor 410 and the coupling unit 420. An extended wire 520 (i.e., a stretchable wire) connects the sensor 410 with the coupling unit 420.

Working range and mechanical properties of the wireless sensors are now described. A feature of the resonance circuit model is that the system works based on a frequency modulation mechanism for chemical sensing by converting changes in electric potential into modulation in capacitance using the varactors, in contrast to an amplitude modulation one relying on tuning the Q factor of the circuit. Although the magnitude of the resonance curve depends on the orientation and distance between the loop antenna and the readout coil due to change in the coupling coefficient, the resonance frequency fₛ represents an intrinsic property of the resonance circuit and is relatively independent of the variation. Thus, it can provide reliable wireless signal transmission despite slight misalignment between the readout coil and the coupling unit. Characterizing electrical performances upon systematically changing the relative position between the coupling unit and the readout coil using a three-axis stage defines the operating range of a coupling unit with an outer diameter (OD)=10 mm, as shown in the readout coil 210 of FIG. 2 for example. In this test, shorting the cathode and anode of the varactors minimizes the impact of environmental noises and static electricity. Test results qualitatively agree with the near-field simulation showing the strength of the magnetic field around the inductor. The mapping of random noise level (defined as “measurement precision”) suggests a minimal impact (i.e., noise <3 mV) of the relative position change on the performance of the sensing platform within the displacement range ((x, y) from (0, 0) to (5, 5), z=5, unit: mm). The value offs also remains almost constant ((x, y)=(0, 0)) with a varying vertical distance between the sensor and the readout coil.

A challenge for building bio-integrated sensors using resonance circuits is that motion/environment-induced noises may cause variations in parasitic reactance, resulting in an undesired shift infs. The use of varactors addresses this issue by dividing the system into the two functional parts connected by extended wires: the coupling unit for wireless signal transmission that can be encapsulated to provide stable electrical performance, and the stretchable sensing interface in direct contact with target fluids/tissues. Placing a damping resistor in series with the sensing interface isolates the bias circuitry from the loop antenna, thereby suppressing parasitic oscillation and minimizing the strain/environment induced changes in electrical properties of the transmission line. The rationale is that the resistance should be high enough for isolating the bias circuitry from the coupling unit without lowering the Q factor.

Interface design and sensing performance in response to various biomarkers is described. FIG. 6 is a schematic illustration of the DC part 600 of ISM-functionalized sensors, and the interaction between ion-selective membranes (ISMs) and ions at the solution-sensor interface. Proper design of the sensing interface coupled to the LC circuit enables the capture, transduction, and readout of biochemical signals. The system successfully realizes the detection of multiple ions by using corresponding ISMs. The interface consists of a sensing electrode (SE) and a reference electrode (RE). Both commercial bulk Ag/AgCl electrodes and self-made thin-film type ones can serve as RE for stable signal readout. The transport of ions from a high to low concentration through a selective binding within the membrane creates a phase boundary potential according to the Nernst equation. It has been determined that the system shows a near-Nernstian sensitivity (49.5±1.5 mV/dec). When connected to the LC circuit, the surface potential difference serves as the reverse bias modulating the capacitance of the varactors, resulting in a shift infs. Following the same working principle, the wireless sensor system can selectively respond to other ions (Na⁺, Ca²⁺, H⁺) in their biologically meaningful ranges via the integration of corresponding ISMs.

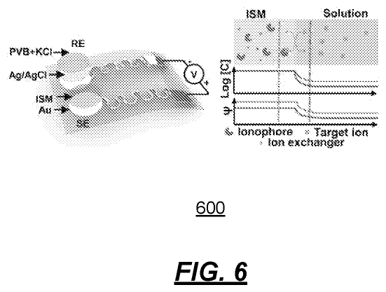

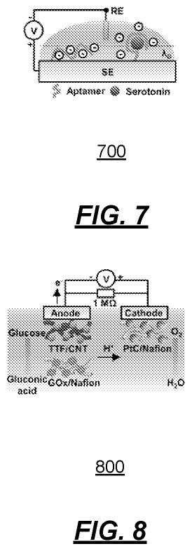

FIGS. 7 and 8 illustrate aptamer and enzyme functionalized sensor systems 700 and 800, respectively. More particularly, FIG. 7 shows a schematic illustration of the interaction between serotonin and an anti-serotonin aptamer-functionalized Au surface, and FIG. 8 shows a schematic illustration of the biofuel cell-inspired biochemical interface for glucose sensing.

Beyond ion sensing, the use of aptamers prepared by the systematic evolution of ligands by exponential enrichment (SELEX) process can expand the applicability of the wireless sensors. Compared to ions with physiologically relevant concentrations at mM or μM level, the concentrations of some biomolecules (e.g., neurotransmitters and hormones) in bodily fluids are naturally much lower (typically at nM or pM level). As a result, minute concentration changes of these biomarkers sometimes fall below the limit of detection (LoD) and limit of quantification (LoQ) of conventional electrochemical sensors when the Faradaic response is small compared to the background current. In this context, SELEX allows for the design of receptors to various analytes. The high affinity between the oligonucleotides and targets provides molecular recognition capability with high sensitivity and selectivity. As an example, FIG. 7 shows the schematic illustration of the interaction between serotonin, a key hormone for emotion regulation, and anti-serotonin aptamers functionalized on a Au electrode surface. The binding event induces a conformational change of the DNA strand, resulting in a modification in surface potential within the Debye screening length, allowing for potentiometric sensing even in an environment with high ionic strength.

The wireless sensor is also compatible with detecting glucose when integrated with a biofuel cell-inspired sensing interface. FIG. 8 illustrates the underlying sensing principle. The anode consists of a highly conductive carbon nanotube paper immobilized with glucose oxidase (GOx) for the catalysis of glucose oxidation. The cathode consists of a current collector built on a platinized carbon-coated gold electrode. The anodic and cathodic reactions generate electrical currents proportional to the concentration of glucose. A load resistor (1 MΩ) connecting the anode and cathode transforms the current into a voltage signal which can be wirelessly transmitted via the LC coupling unit. Optimizing structures of the cathode and anode yields sensors with a near linear dynamic range encompassing the biologically meaningful concentration of glucose in sweat.

The system is suitable for continuous monitoring of glucose in biofluids such as sweat and tears. The system can be easily customized for detection of different analytes using corresponding enzymes, such as lactate, ethanol, urea, and others. Together, the compatibility of this wireless sensor with various types of biochemical interfaces (ISM-, aptamer- and biofuel cell-based) suggests the broad applicability of this device concept.

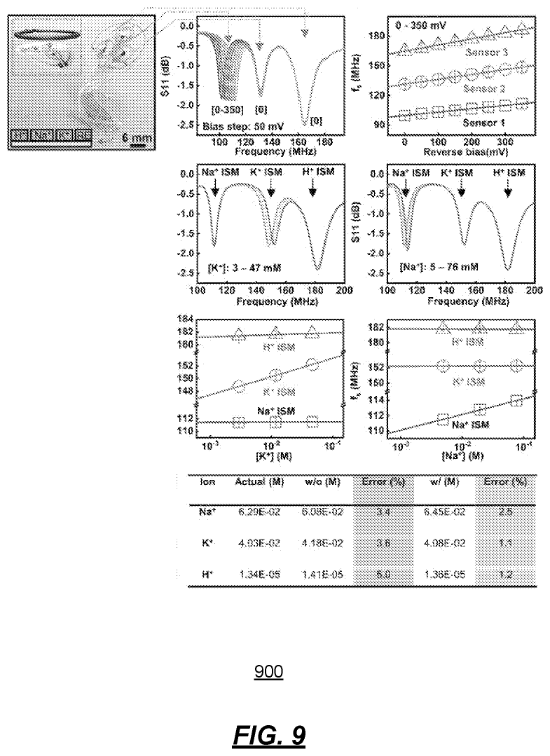

FIG. 9 is a photograph of a multiplexed sensor system 900 based on the LC circuit and sensing performance in mixed ion solutions. More particularly, the photograph is a multiplexed ion sensor system consisting of three LC circuits with varied resonance frequency. The four electrodes (from left to right) correspond to H⁺, Na⁺, K⁺ sensors and the reference electrode.

Another performance metric of biochemical sensors is the multifunctionality which allows for the detection of multiple biomarkers simultaneously with a minimum level of crosstalk. Such multiplexed sensing platforms can support the capture of biometric signature profiles for dynamic, personalized, and adaptive treatments. Designing coils with varied inductance yields well-separated fₛ. As shown in FIG. 9, a two, three, and four turn coil (diameter: 10 mm) possesses a fₛ of 160, 130, and 100 MHz, respectively. The four electrodes in the DC part (from left to right) correspond to H⁺, Na⁺, K⁺ sensors and a shared Ag/AgCl reference electrode. Varying the input voltage to one circuit induces shift in fₛ accordingly, while the values of fₛ of the other two circuits stay almost constant. Based on the frequency modulation sensing mechanism, the multiplexed sensors allow for simultaneous readout of multiple biochemical signals within a single scan. Systematically varying the composition of a mixed solution ([H⁺]=4 M, [Na⁺]=5 mM, [K⁺]=3 mM) and characterizing the response yield a matrix describing the cross-sensitivity of the sensor array. The concentration variation of the base solution takes place according to the following order: [H⁺] from 4 to 16 μM, [Na⁺] from 5 to 76 mM, and [K⁺] from 3 to 47 mM. The sensitivity matrix allows for the calibration of the sensing results with an improved detection accuracy according to the Nikolskii-Eisenman equation. Briefly, the model assumes that the response of a sensor in a complex environment is the sum of responses to specific (Sn) and nonspecific (Sᵢₙ, i≠j) interactions, and calibration using the sensitivity matrix can separate the two types of signals. The results suggest that utilizing the multiplexed sensing platform with the calibration standards can provide an improved accuracy for chemical sensing in complex environment.

Thus, the design is based on the inductor-capacitor (LC) resonance of a coil, a varactor diode, and a damping resistor electrically connected with a functionalized biochemical interface for potentiometric sensing. The multifunctional sensing platform can find broad applications in next-generation healthcare, such as personalized stress monitoring and management.

The wireless and battery-free sensor design is based on the LC resonance of a coil inductor and a varactor diode. A damping resistor placed in series with the biochemical interface can minimize the strain/environment induced changes in properties of the electronic components, enabling the high-fidelity recording in the presence of external strains. The coupling between the sensing interface and the varactor diode converts the change in surface potential due to chemical adsorption into a modification in the capacitance of the varactor diode and then into a shift in the resonance frequency, fₛ. Coupling the inductor to an alternating magnetic field through a readout coil enables the monitoring of the impedance or input return loss using a vector network analyzer. Measuring the impedance or input return loss as a function of frequency yields a peak at fₛ, and fitting the curve determines the value offs. Measuring fₛ in solutions with varied concentration of target analytes yields the calibration curve.

In some implementations, battery-free wireless biochemical sensors are provided. In a resonance circuit, the coupling between a functionalized sensing interface and a LC circuit through a pair of varactor diodes converts a change in electric potential into a modulation in capacitance, which can be quantified by measuring the resonance frequency. Proper design of the sensing interfaces with bio-recognition elements enables the detection of various biomarkers, including ions, neurotransmitters, and metabolites.

Bio-integrated wireless chemical sensors are provided. The system includes an inductor-capacitor (LC) resonance loop antenna consisting of a coil and a pair of varactor diodes electrically coupled with a functionalized biochemical interface. The varactors convert a change in electric potential caused by surface biochemical events into a capacitance modulation, which can then be quantified by reading the shift in the resonance frequency (fₛ) of the LC circuit. Compared to state-of-the-art digital wireless schemes with on-chip integrated circuits and power supply systems, the circuit layout provided herein allows for the miniaturization of the resulting devices, enabling their applications as lightweight, battery-free, and bio-integrated electronics. By properly designing the sensing interfaces using corresponding bio-recognition elements (e.g., ion-selective membranes, aptamers, and enzymes) to induce a potential change proportional to the concentration of biomarkers, this sensing strategy can be customized for detecting a variety of analytes, such as ions, neurotransmitters, and metabolites. As a result, this system can serve as a general and versatile biochemical sensing platform. Compared to conventional resonance circuit based biochemical sensors where the chemically responsive elements are within the LC tank, the varactor diodes divide the system into two functional units: an electromagnetic coupling unit that can be separately encapsulated to provide stable performance in signal transmission, and an extended sensing interface in direct contact with target biofluids. A resistor placed in series with the sensing interface can minimize strain-induced changes in the electrical performance.

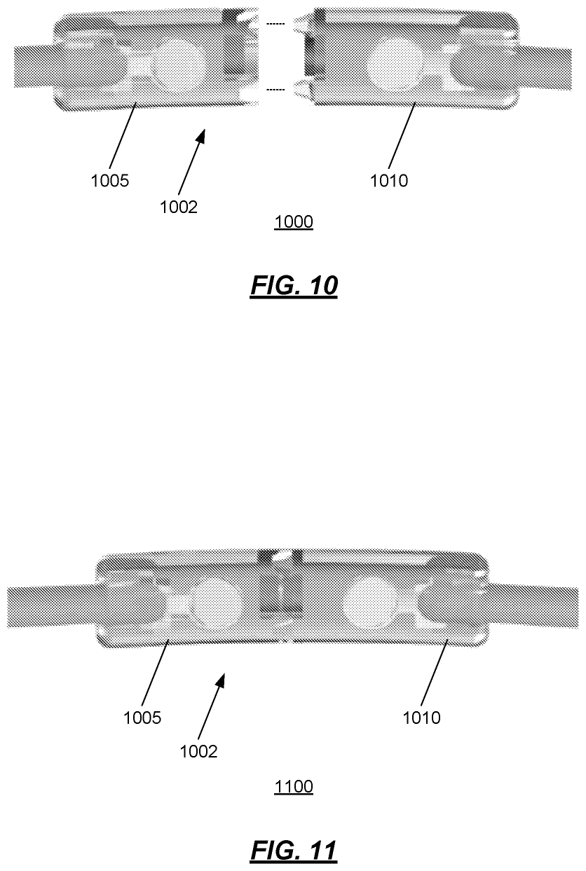

FIGS. 10 and 11 illustrate diagrams of an implementation of a clasp 1002 of a “smart necklace” in an open state 1000 and closed state 1100 that may be worn to monitor a subject's biomarkers in bodily fluids. The clasp 1002 comprises a first end portion 1005 and a second end portion 1010 that may be joined together to form a wearable device.

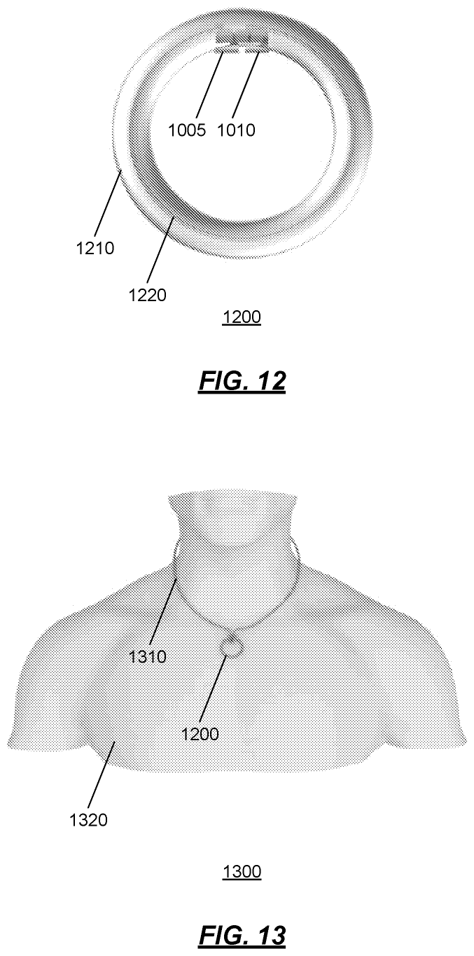

FIGS. 12 and 13 illustrate diagrams of an implementation of a wearable device 1200 (e.g., a pendant of a “smart necklace”) that may be worn by a subject 1300 to monitor the subject's biomarkers in bodily fluids. In an implementation, the wearable device 1200 may comprise the closed clasp 1002 (with the first end portion 1005 and the second end portion 1010 connected by a wire or other connection apparatus 1220 embedded in an enclosure 1210. In an implementation, the wearable device 1200 may be affixed to a chain 1310, for example, and worn on the body 1320 of the subject 1300.

In this manner, a “smart necklace” device is provided and integrated with a functional pendant, clasp, and chain and may be used for real-time monitoring of glucose in sweat or other biomarkers in bodily fluids.

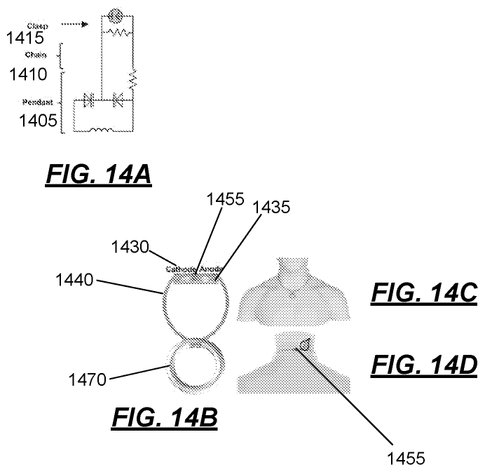

FIGS. 14A, 14B, 14C, and 14D illustrate diagrams of an implementation of a “smart necklace”. FIG. 14A shows a schematic illustration of a “smart necklace” based on the tuning circuit including a “pendant” 1405, a “chain” 1410, and a “clasp” 1415.

As noted further herein, a feature of this tuning circuit inspired sensor system is that it comprises an AC part for wireless signal transmission and DC part for interfacing target biofluids. Customizing this modularized circuit model can form various bio-integrated sensor systems suitable for different application scenarios. For example, the battery-free sensor prototype allows for easy integration with personal accessories due to the light weight and the straightforward circuit layout, providing strategic advantages in health monitoring during daily activities as it does not require the use of on-chip integrated circuits.

In an implementation, encapsulating the sensing interface and the recyclable coupling unit into epoxy scaffolds forms the clasp 1455 (placed on the back of the neck 1460) and pendant 1470 parts, respectively. Here, the clasp 1455 includes a functionalized cathode 1430 and anode 1435 for glucose sensing. Conductive, stretchable wires serve as the chain 1440 connecting the clasp 1455 and pendant 1470. Following the same circuit model, a resistor (incorporated in the “pendant”) in series with the sensing interface can isolate the DC part and minimize motion and/or environment-induced changes in fₛ (e.g., deformation/stretching of the “chain”).

FIGS. 14C and 14D illustrate a human subject wearing the sensor and the key functional parts of the necklace. In an implementations, aligning a single turn readout coil connected with a portable VNA to the “pendant” records the stimulus spectrum within ˜10 s. Analyzing artificial sweat samples using a commercial colorimetric assay kit and the sensor establishes the calibration plot with a Pearson correlation coefficient of 0.9675. Implementing the sensor system in a detachable layout with a recyclable coupling unit and a replaceable sensing interface could provide a viable solution for future remote healthcare due to the reusability and low-cost nature.

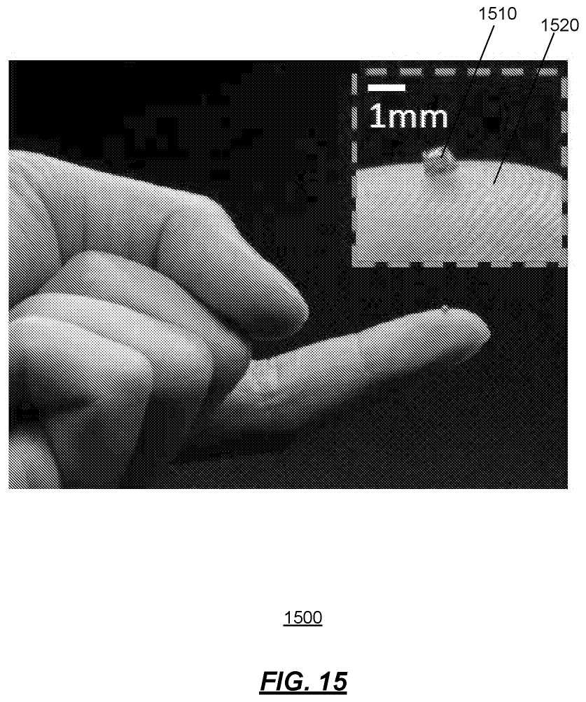

FIG. 15 illustrate a diagram of an implementation 1500 of an implantable sensor 1510 that may be used to monitor a subject's biomarkers. A miniaturized sensor probe based on the resonance circuit may be used as a bioimplant. The implantable sensor 1510 may be implanted into a subject's body, such as in the subject's finger 1520, in an implementation. The sensor 1520 may comprise a miniaturized coupling unit and be mounted on a fingertip.

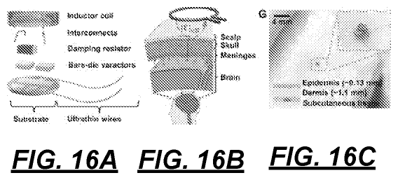

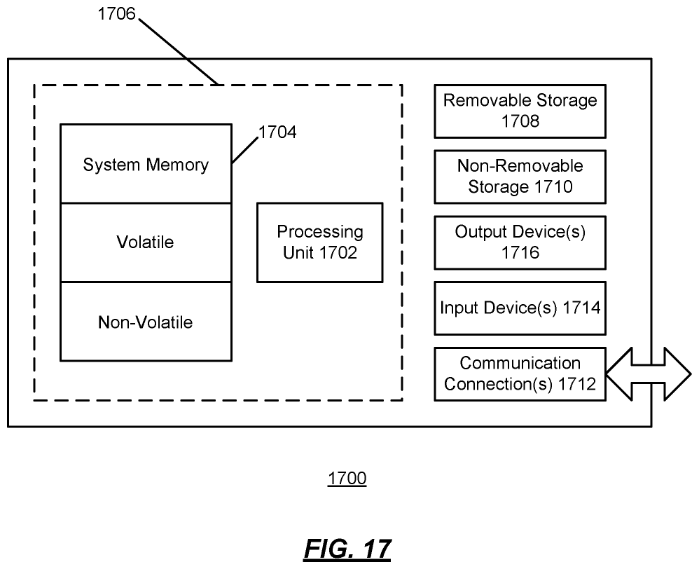

Decapping commercial varactor diodes minimizes the size of the system. Wrapping a pair of diodes with ultrathin conductive wires forms the inductor coil. FIG. 16A shows an exploded view of an as-prepared probe and FIGS. 16B and 16C show conceptual illustrations of a potential application where the coupling unit is embedded in the skull to provide stable signal transmission while the sensing interface extends to the area with cerebrospinal fluid (CSF) or brain tissues of interest.

It is noted that conventional resonance circuit based biochemical sensors typically have a functionalized electronic component integrated within the LC tank, the resistance/capacitance of which can be modulated upon chemical adsorption. However, the part interfacing with bio-tissues may affect the performance of the transmitter and thus can frustrate the quantitative interpretation of biochemical signals. In the systems and methods provided herein, the separation of the sensing interface from the coupling unit can enable the placement of the AC and DC parts into different locations with ultrathin wires as interconnects and the resistor minimizing motion/environment induced artifacts in the DC part. Moreover, compared with the conventional Bluetooth and NFC wireless schemes, the systems and methods provided herein minimize the use of rigid components which significantly improves the conformability for bio-integration.

Further miniaturizing the circuit model to form probe type sensors is possible for potential applications such as implantable devices.

Thus, by wirelessly measuring biomarkers concentrations associated with the response of the body to the environment, stress, and diseases, the systems and methods described herein can find broad applications in biomedical research and clinical practices.

In summary, the materials, electronic designs, and integration schemes presented herein provide a promising route to developing miniaturized, lightweight, wireless, and bio-integrated technologies. The resulting system combines LC resonance circuit, varactor diode, stretchable design, and bio-recognition elements for detection of biological markers in bodily fluids. Inspired by the principle of tuning circuits in RF electronics, the coupling between LC circuits and sensing interfaces through a pair of varactor diodes enables battery-free operation and accurate monitoring of multiple biomarkers with a straightforward circuit layout. Specifically, the use of varactor diodes modularizes the system into an AC part (coupling unit) that can be encapsulated separately and a DC part (sensing interface) in contact with target biofluids. The integration of a series resistor with the DC part can minimize strain induced changes in electrical properties of the circuit, enabling the high-fidelity recording in the presence of external strains. Sensors with ISM-, aptamer-, and enzyme-functionalized interfaces suggest that this sensing platform can support the detection of ions, neurotransmitters, and metabolites via proper design of the surface structure and coupling strategy. A “smart necklace” built on the circuit model demonstrates that these devices can implement robust signal recording and data transmission without failure during physical exercise. Overall, the sensing platform provides strategic advantages for recording, tracking, and understanding the body's dynamic chemistry. Devices of this type can serve as a complementary tool for health status tracking and close-loop health management to commercial wearable sensors that record biophysical signals. By wirelessly tracking biomarker concentrations associated with the response of the body to environment, stress, and disease, this innovative, versatile device concept can find broad applications in biomedical research and clinical practices. Immediate opportunities are with developing lightweight, miniaturized, and portable readout circuits to replace the commercial bulky VNA for data transmission to user interfaces, which can be easily integrated with the user's personal belongings, such as clothes and cell phones for remote health monitoring.

An implementation of fabrication of an LC-circuit based sensing platform is described and is not intended to be limiting. The fabrication of the electromagnetic coupling unit started with laminating a conductive copper tape (adhesive side facing up) on an ultraviolet (UV) light dicing tape. Cutting the copper tape using a vinyl cutter followed by exposing the system to UV light and peeling off unneeded parts of the tape yielded patterned conductive traces. Pasting the adhesive side of the copper tape on a polyester (PET) film and removing the UV dicing tape finished the transfer process. Soldering a pair of varactor diodes (SMV1249-040LF, Skyworks Solutions, Inc., CA, USA) and a 10 kW resistor to the metal traces formed the LC circuit. The preparation of the electrodes as the sensing interface followed the same procedures, except that Cr (10 nm)/Au (300 nm) deposited on a polyimide (PI) film (˜13 μm) using electron-beam evaporation served as the conductive metal traces which provided stable signal readout due to the chemical inertness of Au. Connecting the DC and AC components using silver epoxy completed the fabrication of the stretchable electronic device.

In an implementation, the preparation of ISM-based sensing interfaces for Na⁺, K⁺, Ca²⁺, H⁺ can be performed using any well-known technique(s). In an implementation and not intended to be limiting, the recipe for each membrane is as follows: (1) Na⁺-ISM: sodium ionophore X (1 wt %), sodium tetrakis[3,5-bis(trifluoromethyl)-phenyl] borate (Na-TFPB, 0.55 wt %), polyvinyl chloride (PVC, 33 wt %), and bis(2-ethylhexyl) sebacate (DOS, 65.45 wt %). Dissolving 100 mg of the mixture in 660 μL tetrahydrofuran formed the Na⁺ ISM cocktail. (2) K⁺-ISM: valinomycin (2% wt %), sodium tetraphenylborate (Na-TPB 0.5 wt %), PVC (32.7 wt %), and DOS (64.7 wt %). Dissolving 100 mg of the mixture in 350 μL of cyclohexanone formed the K⁺ ISM cocktail. (3) Ca²⁺-ISM (directly purchased from Sigma Aldrich): calcium ionophore IV (0.072 wt %), Na-TFPB (0.022 wt %), 2-nitrophenyl octyl ether (4.748 wt %), PVC (2.379 wt %) and tetrahydrofuran (92.78 wt %). (4) H⁺-ISM: hydrogen ionophore I (1 wt %), Na-TClPB (0.65 wt %), PVC (33 wt %), and DOS (65.35 wt %). Dissolving 100 mg of the mixture in 660 μL tetrahydrofuran formed the H⁺ ISM cocktail. Casting the mixtures on Au surface and drying the system overnight completed the preparation of ISM-functionalized sensing interface.

An implementation of a preparation of thin-film Ag/AgCl reference electrode is described and is not intended to be limiting. The preparation of the reference electrode starts with drop-casting a mixture of silver epoxy and hardener (e.g., Chemtronics CW2400) on a Au electrode surface and curing it at room temperature for 12 hours. Treating the electrode with sodium hypochlorite solution (5 wt %) for 30 min converts the surface to AgCl. In a separate process, recrystallizing KCl(aq) in cold isopropyl alcohol (IPA) forms ultrafine micro-size powders. Dissolving 438 mg polyvinyl butyral (PVB, 10 wt %) in 5 mL anhydrous ethanol, mixing the solution with 250 mg KCl powder, and homogenizing the system in an ultrasonic bath for 10 min yields an electrolyte cocktail (stored at 7° C.). Casting the electrolyte cocktail on the Ag/AgCl electrode and drying the whole system overnight completes the fabrication of the reference electrode.

An implementation of a preparation of aptamer-based sensing interface for serotonin is described and is not intended to be limiting. Thiolated, single stranded anti-serotonin DNA aptamer (5′ to 3′: HO(CH₂)₆—S—S—(CH₂)₆-CTC TCG GGA CGA CTG GTA GGC AGA TAG GGG AAG CTG ATT CGA TGC GTG GGT CGT CCC) was obtained from Integrated DNA Technologies (IDT, Coralville, IA). The DNA aptamer received from the manufacturer was in an oxidized state with S atoms protected in the form of disulfide bonds. Reducing the oxidized DNA aptamer with dithiothreitol (DTT) (100 μM DNA aptamer, 10 mM DTT in 1× Tris-ethylenediaminetetraacetic acid (TE) buffer) followed by removing DTT through centrifugation in Macro SpinColumns (Harvard Apparatus) yielded DNA aptamer with thiol groups on the 5′ ends for binding to Au surfaces. Mixing the aptamer (50 μM) and 6-mercapto-1-hexanol (MCH) (1:100) in 1× TE buffer formed the coating solution for Au surface. Prior to functionalization, treating the Au electrode by cyclic voltammetry in 0.5 M H₂SO₄ (−0.4˜1.5 V vs Ag/AgCl) for 3 cycles cleaned the surface. Drop-casting the mixture onto the cleaned Au electrode and drying the system at room temperature overnight formed Au—S bonds and thereby completed the functionalization. Before testing, immersing the sensing electrode in 1 mM MCH solution for 30 min further passivated any remaining exposed Au area. After an incubation period of 30 min in PBS solutions with different concentration of serotonin, an electrochemical working station (PalmSens4, CA, USA) recorded the open circuit potential (OCP) (vs. Ag/AgCl reference electrode) with a sampling rate of 1 Hz. The characterization of electrochemical impedance spectroscopy (EIS) used a three-electrode set up with a Ag/AgCl reference electrode and a platinum wire counter electrode. K₄Fe(CN)₆/K₃Fe(CN)₆ (1:1) (2 mM) served as the redox couple. Simulation of the measured Nyquist plots using the Z-view software based on a Randles circuit determined the charge transfer resistance (Ret).

An implementation of a preparation of the biofuel-based sensing interface for glucose is described and is not intended to be limiting. The preparation of biofuel cell-based sensors followed previous studies. The enzyme-modified anode consisted of tetrathiafulvalene (TTF), GOx, bovine serum albumin (BSA), and Nafion 117. Dissolving TTF (Sigma Aldrich, MO, USA) in a mixture of ethanol/acetone (9:1, v/v) and homogenizing it in an ultrasonic bath for 10 min formed uniform solution (0.1 M). Drop-casting 32 μl of TTF solution onto a multi-walled carbon nanotube (MWNT) paper (100 mm²; Buckypaper, GSM 20; NanoTechLabs, NC, USA) and letting dry for 1 hour under ambient condition formed TTF modified carbon nanotube pads. Dispersing GOx (40 mg/ml; Sigma Aldrich, MO, USA) and BSA (10 mg/ml; Sigma Aldrich, MO, USA) in 1×PBS and 1×PBS with Nafion 117 (Sigma Aldrich, MO, USA) (1 wt %), respectively, followed by mixing the two suspensions in equal volumes, yielded the enzyme-coating suspension. Applying 64 μL of the enzyme-coating suspension onto the TTF treated MWNT paper, letting dry for 1 hour under ambient condition, and storing it at 7° C. for a week allow the system to equilibrate. Finally, using a mechanical puncher to cut an enzyme-functionalized pad (diameter: 1 mm) and bonding it to a gold current collector with silver epoxy completed the fabrication of the anode.

An implementation of the preparation of the cathode, and not intended to be limiting, started with dispersing platinum on carbon (Pt/C, 10%; Sigma-Aldrich, MO, USA) in a Nafion solution (2 wt % in ethanol). Applying 5 μL of the resulting suspension (10 mg/ml in Nafion solution) on a MWNT paper (1 mm²), drying the solution under ambient condition for 1 hour, and storing it at 7° C. for a week formed the functionalized cathode. Bonding the Pt/C modified pad to a gold current collector with silver epoxy completed the fabrication. Exposing both the cathode and anode to 1×PBS for 30 min before testing allowed the system to reach equilibrium providing stabilized signals.

An implementation of fabrication of a “smart necklace” and miniaturized probe is described and is not intended to be limiting. The fabrication of the pendant in a smart necklace followed the same steps for preparing the LC circuit. Encapsulating the LC circuit with waterproof clear epoxy formed the pendant with two exposed contact pads. Weaving a flexible conductive copper wire with two strings of nylon wire yielded the chain. Depositing Cr (10 nm)/Au (300 nm) on a polyimide (PI) film (˜13 μm) and transferring it onto a PET film formed the current collectors for the clasp. Subsequently, bonding an enzyme modified MWNT pad and a Pt/C modified pad to the corresponding current collectors formed the anode and cathode, respectively. A 1 MΩ load resistor was bonded to the Au traces by silver epoxy. Finally, electrically connecting the pendant, chain, and clasp via soldering followed by encapsulating the junctions with waterproof epoxy completed the fabrication of the smart necklace.

An implementation of fabrication of the miniaturized probe, not intended to be limiting, started with depositing metal traces (Cr (10 nm)/Au (300 nm)) onto a PET film as electrical interconnects using electron-beam evaporation and a shadow mask. A mechanical puncher defined 1 mm PET pad as the substrate for the coupling unit. Bonding a pair of decapped bare-die varactor diodes (SMV1249, Skyworks), a thick-film resistor (10 KΩ, size=01005), jumping wires (Cu, wire diameter=50 μm), and a manually wrapped inductor coil (Cu, coil diameter=1 mm, 11 turns, wire diameter=50 μm) to the deposited metal traces formed the miniaturized LC circuit. Encapsulating the whole device with waterproof UV-curing resin (NOA 61, Norland Products, USA) completed the fabrication.

An implementation of data collection and analyses is described and is not intended to be limiting. The readout circuit consists of a single turn coil connected to a portable vector network analyzer (NanoVNA) through a Sub-Miniature Version A (SMA) cable. Setting the VNA to reflective mode enables the measurement of the real and imaginary part of the reflection coefficient. Expressing the raw data in dB yields S11. Sweeping the frequency range obtained the attenuation of the coupling unit around fₛ.

FIG. 17 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 17, an exemplary system for implementing aspects described herein includes a computing device, such as computing device 1700. In its most basic configuration, computing device 1700 typically includes at least one processing unit 1702 and memory 1704. Depending on the exact configuration and type of computing device, memory 1704 may be volatile (such as random access memory (RAM)), non-volatile (such as read-only memory (ROM), flash memory, etc.), or some combination of the two. This most basic configuration is illustrated in FIG. 17 by dashed line 1706.

Computing device 1700 may have additional features/functionality. For example, computing device 1700 may include additional storage (removable and/or non-removable) including, but not limited to, magnetic or optical disks or tape. Such additional storage is illustrated in FIG. 17 by removable storage 1708 and non-removable storage 1710.

Computing device 1700 typically includes a variety of computer readable media. Computer readable media can be any available media that can be accessed by the device 1700 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 1704, removable storage 1708, and non-removable storage 1710 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1700. Any such computer storage media may be part of computing device 1700.

Computing device 1700 may contain communication connection(s) 1712 that allow the device to communicate with other devices. Computing device 1700 may also have input device(s) 1714 such as a keyboard, mouse, pen, voice input device, touch input device, etc. Output device(s) 1716 such as a display, speakers, printer, etc. may also be included. All these devices are well known in the art and need not be discussed at length here.

In an implementation, a system comprises: a sensing interface comprising an inductor-capacitor (LC) circuit configured to detect multiple biomarkers in bodily fluids of a subject; a coupling unit; and a stretchable wire connecting the sensing interface and the coupling unit.

Implementations may include some or all of the following features. The sensing interface is configured to use the LC resonance of a coil and a varactor diode electrically connected with a functionalized biochemical interface for potentiometric sensing. The LC circuit comprises at least one of a capacitor or a varactor configured to control a working frequency band. The sensing interface comprises at least one type of potentiometric bio-recognition element. The at least one least one type of potentiometric bio-recognition element comprises at least one of metal oxides, ion-selective membranes, aptamers, antibodies, molecularly imprinted polymers (MIPs), and/or enzymes. The system further comprises a damping resistor in series with the sensing interface and configured to minimize strain or environment induced changes in electronic properties of the LC circuit. The bodily fluids comprise at least one of sweat, cerebrospinal fluid, blood, plasma, interstitial fluid, saliva, tear, breast milk, amniotic fluid, mucus, gastric acid, or urine. The biomarkers comprise at least one of antigens, metabolites, neurotransmitters, hormones, electrolytes, or cells. The sensing unit, the coupling unit, and the stretchable wire are comprised within a wearable device. The sensing unit, the coupling unit, and the stretchable wire are comprised within a biomedical implant. The system is configured to monitor at least one of a physiological status or a pathological status of the subject without battery and configured to transmit data wirelessly. The system is configured to transmit data via a frequency modulation mechanism. The system further comprises a receiver and a transducer, wherein the transducer comprises the coupling unit, the stretchable wires, and a potentiometric sensor. The coupling unit comprises multiple LC circuits configured with separated resonance frequency bands.

In an implementation, a system comprises: a receiver and a transducer configured to detect multiple biomarkers simultaneously in body fluid of a subject and monitor a health status of the subject without a battery and configured to transmit data wirelessly.

Implementations may include some or all of the following features. The transducer comprises multiple biochemical sensors functionalized with different bio-recognition elements. The biochemical sensors are configured to transmit signals through different coils with separated resonance frequency bands.

In an implementation, a method comprises: determining an electric potential change in a DC part of a LC resonance chemical sensor; determining a capacitance modulation in an AC part; and determining a resonance frequency shift in the AC part.

Implementations may include some or all of the following features. The method further comprises detecting a biomarker in body fluid of a subject using the electric potential change and the LC resonance chemical sensor. The method further comprises monitoring at least one of a physiological status or a pathological status of the subject.

As used herein, the singular form “a,” “an,” and “the” include plural references unless the context clearly dictates otherwise. As used herein, the terms “can,” “may,” “optionally,” “can optionally,” and “may optionally” are used interchangeably and are meant to include cases in which the condition occurs as well as cases in which the condition does not occur.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A system comprising:
a sensing interface comprising an inductor-capacitor (LC) circuit configured to detect multiple biomarkers in bodily fluids of a subject;
a coupling unit; and
a stretchable wire connecting the sensing interface and the coupling unit.

2. The system of claim 1, wherein the sensing interface is configured to use the LC resonance of a coil and a varactor diode electrically connected with a functionalized biochemical interface for potentiometric sensing.

3. The system of claim 1, wherein the LC circuit comprises at least one of a capacitor or a varactor configured to control a working frequency band.

4. The system of claim 1, wherein the sensing interface comprises at least one type of potentiometric bio-recognition element.

5. The system of claim 4, wherein the at least one least one type of potentiometric bio-recognition element comprises at least one of metal oxides, ion-selective membranes, aptamers, antibodies, molecularly imprinted polymers (MIPs), and/or enzymes.

6. The system of claim 1, further comprising a damping resistor in series with the sensing interface and configured to minimize strain or environment induced changes in electronic properties of the LC circuit.

7. The system of claim 1, wherein the bodily fluids comprise at least one of sweat, cerebrospinal fluid, blood, plasma, interstitial fluid, saliva, tear, breast milk, amniotic fluid, mucus, gastric acid, or urine.

8. The system of claim 1, wherein the biomarkers comprise at least one of antigens, metabolites, neurotransmitters, hormones, electrolytes, or cells.

9. The system of claim 1, wherein the sensing unit, the coupling unit, and the stretchable wire are comprised within a wearable device.

10. The system of claim 1, wherein the sensing unit, the coupling unit, and the stretchable wire are comprised within a biomedical implant.

11. The system of claim 1, wherein the system is configured to monitor at least one of a physiological status or a pathological status of the subject without battery and configured to transmit data wirelessly.

12. The system of claim 1, wherein the system is configured to transmit data via a frequency modulation mechanism.

13. The system of claim 1, further comprising a receiver and a transducer, wherein the transducer comprises the coupling unit, the stretchable wires, and a potentiometric sensor.

14. The system of claim 1, wherein the coupling unit comprises multiple LC circuits configured with separated resonance frequency bands.

15. A system comprising:
a receiver and a transducer configured to detect multiple biomarkers simultaneously in body fluid of a subject and monitor a health status of the subject without a battery and configured to transmit data wirelessly.

16. The system of claim 15, wherein the transducer comprises multiple biochemical sensors functionalized with different bio-recognition elements.

17. The system of claim 16, wherein the biochemical sensors are configured to transmit signals through different coils with separated resonance frequency bands.

18. A method comprising:
determining an electric potential change in a DC part of a LC resonance chemical sensor;
determining a capacitance modulation in an AC part; and
determining a resonance frequency shift in the AC part.

19. The method of claim 18, further comprising detecting a biomarker in body fluid of a subject using the electric potential change and the LC resonance chemical sensor.

20. The method of claim 19, further comprising monitoring at least one of a physiological status or a pathological status of the subject.
